(19) United States
(12) Patent Application Publication
(10) Pub. No.: US 2012/0309000 A1
(43) Pub. Date: Dec. 6, 2012

(54) MYCOBACTERIA-DERIVED DNA MISMATCH REPAIR NUCLEOTIDE SEQUENCES AND USES THEREOF

(75) Inventors: Bum-Joon Kim, Seoul (KR); Byoung-Jun Kim, Uiwang-si (KR)

(73) Assignee: SNU R&DB Foundation, Seoul (KR)

(21) Appl. No.: 13/467,227

(22) Filed: May 9, 2012

(30) Foreign Application Priority Data
May 9, 2011 (KR) 10-2011-0043380

(51) Int. Cl. C12N 15/61; C12Q 1/68; C12N 1/21; C12N 15/113; C12N 15/74

(52) U.S. Cl. 435/6.11; 536/23.2; 536/24.1; 435/320.1; 435/252.3; 435/6.15; 435/6.12; 435/471; 435/476

(57) ABSTRACT

The present disclosure provides an isolated DNA molecule derived from Mycobacteria having a DNA mismatch repair function represented by a nucleic acid sequence as disclosed in SEQ ID NO: 1 or 2. Also provided is a promoter sequence having SEQ ID NO: 3, and a recombinant vector comprising the isolated DNA of the present disclosure and a promoter operatively linked to the DNA molecule. The isolated DNA molecule of the present disclosure is classified as MutS4A and MutS4 and confers cells with resistance to UV and macrophages when transformed into the cells. Further it increases the frequency of homologous recombination and the genetic stability of a heterologous plasmid in cells.

### CROSS-REFERENCE TO RELATED APPLICATIONS

This application claims the benefit of Korean Patent Application No. 10-2011-0043380, filed MAY 9, 2011, in the Korean Intellectual Property Office, the disclosure of which is incorporated herein by reference.

### BACKGROUND OF THE INVENTION

1. Field of the Invention

The present disclosure relates to DNA mismatch repair nucleotide sequences derived from Mycobacteria and their use.

2. Description of the Related Art

DNA mismatch repair (MMR) is a cellular system for recognizing and repairing DNA polymerase errors that escape detection in proofreading and improves the replication fidelity by 50 to 1000 times [1,2]. Much of the research in this area has been done using the E.coli MMR systems, where a MutS homodimer binds to mismatched base pairs to form a MutS-DNA complex and then is joined by a MutL homodimer in an ATP dependent manner. The complex formed then activates an endonuclease MutH and cut out the mismatched base and replace it with a correct base [1,2].

Various genes homologous to E.coli MutS have been found in bacteria, archaea and even in eukaryotic cells [3].

Mycobacteria are mainly divided into two groups of absolute tuberculosis mycobacteria and opportunistic non-tuberculosis mycobacteria that are commonly found in the environment. They belong to a highly clonal population generating genetic mutations through gene rearrangement rather than later gene transfer (LGT). It is known that the tuberculosis mycobacteria acquire the resistance to antibiotics only through chromosomal mutations [4], the reason of which has been thought that they do not have the MMR system.

The function of MMR system in cells is to reduce the rate of genetic mutation such as duplication and to correct the heteroduplex DNA intermediate which is the by-product of a recombination event [5,6]. Therefore, it was inferred that the absence of MMR system in Mycobacteria has caused them to depend on the genetic mutations such as duplication and paralogue formation as their main drive for genetic changes, and has resulted in the less recombination event.

Previously known DNA mismatch repair genes from Mycobacteria belong to a MutS4 form among the homologues of MutS from E.coli and. MutS4 genes include MutS4A and MutS4B. They are present on the genome adjacent to each other and the stop codon of MutS4A overlaps with the initiation codon of MutS4B [3]. This genetic structure is also found in other bacteria and archaea, which has been thought to be the result of the two MutS4 genes produced through tandem duplication and later transferred to other bacteria or archaea through horizontal gene transfer.

### SUMMARY OF THE INVENTION

In the present disclosure, there are provided two novel genes, classified as MutS4A and MutS4B, which have been found to have a high sequence homology to DNA mismatch repair protein from other species and have conferred the mycobacterial transformed with the present genes with resistance to UV and macrophage. The present genes were derived from a novel Mycobacterial species, named Mycobacterium yongonense (05-1390T) (DSM (German Collection of Microorganisms and Cell Cultures) 45126T, KCTC (Korean Collection for Type Culture) 19555T).

In one aspect, the present disclosure provides an isolated nucleic acid molecule derived from a newly identified mycobacteria having a DNA mismatch repair function and having a nucleic acid sequence as disclosed in SEQ ID NO:1 or NO: 2.

In yet another aspect, the present disclosure provides a promoter having a nucleic acid sequence as disclosed in SEQ ID NO: 3. In one embodiment the promoter is used for regulating the expression of a gene that confers a cell with UV resistance.

In still other aspect, the present disclosure provides a recombinant vector comprising (i) the DNA molecule of the present disclosure; and (ii) a promoter operatively linked to the DNA.

In still other aspect, the present disclosure provides a cell transformed with the present vector as disclosed herein.

In still other aspect, the present disclosure provides a method of identifying a MOTT (mycobacteria other than tuberculosis), particularly MOTT related to M.intracellulare INT-5, M. yongonense, MOTT-12, MOTT-27 or MOTT64y in a sample by detecting the isolated DNA molecule of the present disclosure.

In still other aspect, the present disclosure provides a kit for diagnosing a disease related to a MOTT comprising a probe and/or a primer set to detect the genes involved in the mismatch repair function in cells. In one embodiment the kit is used to detect the isolated DNA molecule of the present disclosure. In one embodiment, the MOTT is a MOTT related to M.intracellulare INT-5, M. yongonense, MOTT-12, MOTT-27 or MOTT64y.

In still other aspect, the present disclosure provides a method of using the isolated DNA molecule of the present disclosure to increase a frequency of homologous recombination.

In still other aspect, the present disclosure provides a method of using the isolated DNA molecule of the present disclosure to increase a genetic stability of a heterologous plasmid in cells.

The foregoing summary is illustrative only and is not intended to be in any way limiting. Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is an amino acid sequence homology tree for MutS4A and MutS4B.

FIG. 2 is a schematic view showing the location of the primer pair in MutS4B for detecting opportunistic non-tuberculosis mycobacterium. DR-MSF: 5′-TCCAGGTCCGGCGCAAGGTGTT-3′; and DRMSR: 5′-CGCGGGCGGCTGATGAAGAAGATA-3′

FIG. 3 is a result of the assay performed to detect Muts4B present in various opportunistic non-tuberculosis mycobacteria (MOTT) using the primer pair as indicated in FIG. 2, where MOTT-90 indicates Mycobacterium yongonense (05-1390), and MOTT-12, MOTT-27, MOTT-64, h2 and h4 indicate Mycobacteria related to Mycobacterium intracellulare INT5 isolated from patient samples, and w and y each indicates white colony and yellow colony from the same patient. Each lane indicates: M: Marker; Lane 1: MOTT-90; Lane 2: MOTT-12; Lane 3: MOTT-27; Lane 4: Mycobacterium intracellulare; Lane 5: h2; Lane 6: h4 (w); Lane 7: h4(y); Lane 8: MOTT-64(w); Lane 9: MOTT-64(y); Lane 10: MOTT-36(w); Lane 11: MOTT-36(y); Lane 12: Mycobacterium bovis BCG; and Lane 13: negative control.

FIG. 4 is a schematic representation of the structure of the DNA mismatch repair construct comprising MutS4A and MutS4B and the promoter, the sequence of which is disclosed as SEQ ID NO: 4.

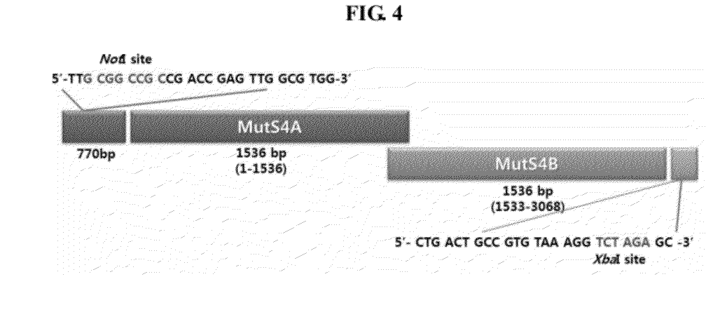

FIG. 5 is a map of the pMV306 vector used for the construction of a recombinant vector comprising the DNA mismatch repair gene of the present disclosure.

FIGS. 6A and 6B are the results of the assay to confirm the transformation of Mycobacterium smegmatis MC2-155 (DRP1 and DRP2) by pMV306 comprising the isolated DNA of the present disclosure using PCR (6A) and RT-PCR (6B). Each lane indicates: M: marker; N: negative control; P: positive control; Lane 1: positive control; Lane 2: Mycobacterium intracellulare; Lane 3: Smeg-pMV306; and Lane 4: Smeg-DNA mismatch repair.

FIG. 7 is a result of the UV resistance assay of Mycobacterium smegmatis transformed with pMV306 comprising the isolated DNA of the present disclosure. The transformed Mycobacteria were irradiated with UV for the indicated time period (0, 3, and 5 min) and then incubated for 2-3 days. The result confirms that Mycobacteria (DRP1 and DRP2) transformed with the pMV306 comprising the isolated DNA of the present disclosure have a higher survival rate compared to that of Mycobacteria transformed with an empty vector pMV306.

FIG. 8 is a result of the assay to test the resistance of Mycobacterium smegmatis transformed with pMV306-DNA of the present disclosure to macrophages. The result confirms that Mycobacteria (DRP1 and DRP2) transformed with the pMV306-DNA of the present disclosure have 3 to 4 times higher resistance to macrophage compared to that of Mycobacteria transformed with an empty vector pMV306.

FIG. 9 is a result of the assay to test the frequency of homologous recombination. The graph shows the number of colonies formed on a medium containing rifampin from M. smegmatis which were transformed with each plasmid indicated.

FIG. 10 is a multiple sequence alignment of rpoB genes isolated from the colonies grown on a medium containing rifampin.

FIG. 11 is a map of the plasmid TOPO 05-1390-EGFP for the FAGS analysis.

FIG. 12 is a result of the FACS analysis to test the changes in the expression level of EGFP in M. smegmatis through multiple generations.

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

In the present disclosure, there were identified two novel genes classified as MutS4A and MutS4B, which have been found to have a high sequence homology to DNA mismatch repair protein from other species. The genes identified in the present disclosure have a DNA mismatch repair function and have conferred the cells with resistance to UV and macrophage when it was transformed to the cells.

Thus, in one aspect, the present disclosure relates to an isolated nucleic acid molecule derived from mycobacteria having a DNA mismatch repair function. The sequence is represented by SEQ ID NO: 1 or 2.

The genus mycobacterium includes pathogens known to cause serious diseases in mammals including tuberculosis (Mycobacterium tuberculosis) and leprosy (Mycobacterium leprae), opportunistic non-tuberculosis mycobacterium, and saprophytic species, and currently a total of about 150 species have been known. (see http://www.bacterio.cict.fr/m/mycobacterium.html). Among those, 25 species have been found to be associated with human disease. The genome project of 17 species out of the 25 has been completed.

The isolated DNA molecule newly identified in the present disclosure was derived from M. yongonense and has a nucleic acid sequence as disclosed in SEQ ID NO:1 or 2. The DNA molecule of the present disclosure may be used for detecting opportunistic non-tuberculosis mycobacterium in a sample.

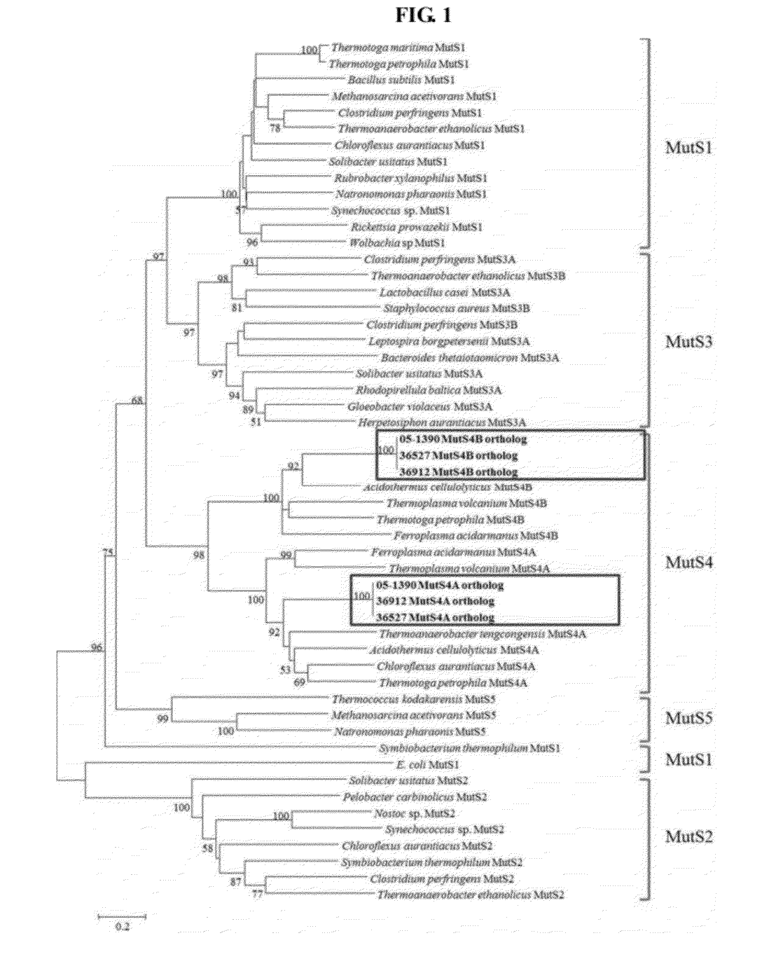

The isolated DNA molecule of the present disclosure has a DNA mismatch repair function and has been classified as MutS4A and MutS4B based on the sequence homology (See FIG. 1). According to the phylogenetic analysis performed based on the amino acid sequences encoded by the present DNA molecule, it has showed the highest homology to MutS4 genes (See FIG. 1, Tables 1 and 2). This indicates that the DNA molecule identified in the present disclosure is a novel MutS4A and MutS4B genes derived from mycobacteria having a DNA mismatch repair function.

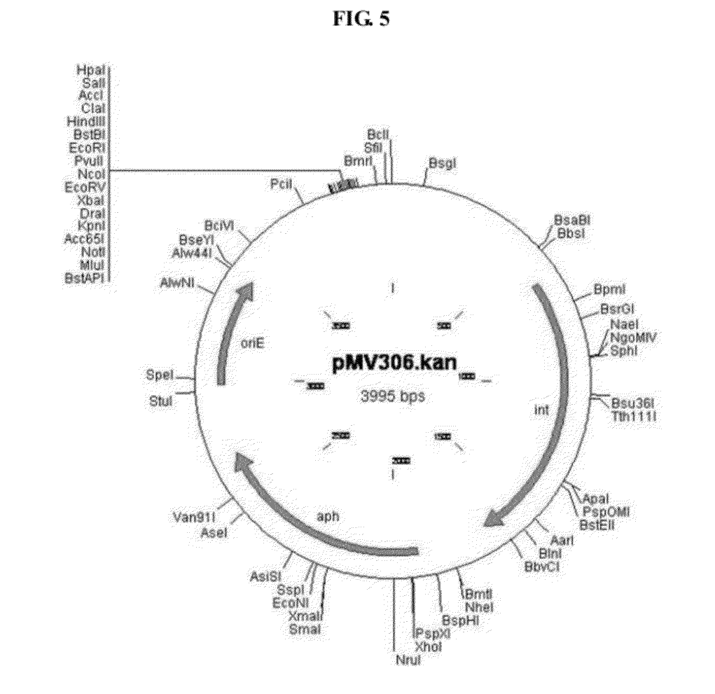

In other aspect the present disclosure relates to a recombinant vector comprising (i) the nucleic acid molecule DNA molecule of the present disclosure, i.e., MutS4A of SEQ ID NO: 2 and/or MutS4B of SEQ ID NO: 2; and (ii) a promoter operatively linked to the DNA. In one embodiment, the vector comprises the DNA molecule of the present disclosure in the pMV306 backbone (See FIG. 5), but is not limited thereto.

The present vector may be constructed as a cloning or expression vector. In addition, the present vector may be constructed to be used in prokaryotic or eukaryotic cells as a host. In one embodiment, the vector is for prokaryotic cells considering that the DNA molecule of the present disclosure is derived from mycobacteria and the culture conditions. For example, when the host cell used is a prokaryotic origin, the vector comprises a strong promoter for transcription such as tac promoter lac promoter, lacUV5 promoter, lpp promoter, pL^{λ} promoter, pR^{λ} promoter, rac5 promoter, amp promoter, recA promoter, SP6 promoter, trp promoter and T7 promoter, and a ribosomal binding site, and a termination sequence for transcription/translation. When the E.coli is used, the promoter and operator region involved in the tryptophan biosynthesis (Yanofsky, C., J. Bacteriol.,158:1018-1024(1984)) and pL^{λ} promoter from phage (Herskowitz, I. and Hagen, D., Ann. Rev. Genet., 14:399-445(1980)) may be used as regulating sequences.

The term “promoter” as used herein indicates DNA sequences which regulate the expression of sequences encoding a protein or a functional RNA. The nucleic acid sequences encoding a target material to be expressed are operatively linked to the promoter. The term “operatively linked” as used herein indicates a functional link between a regulatory sequence for the expression of nucleic acids including, for example, promoter sequences, signal sequences, or transcription factor binding site, and other nucleic acid sequences. Here the regulatory sequence regulates the transcription or translation of the other nucleic acid sequences linked thereto.

In one embodiment, the promoters which may be used for the present vector include, but are not limited to, a promoter according to the present disclosure having a nucleic acid sequence as disclosed in SEQ ID NO: 3, a heat shock protein promoter, a CMV promoter, a promoter for 65 kDa common antigen of mycobacteria, a ribosome RNA promoter from Mycobacteria, a promoter for MPB70, MPB59 or MPB64 antigen from Mycobacterium bovis, tac promoter, trp promoter, lac promoter, lacUV5 promoter, P_{L}^{λ} promoter, P_{R}^{λ}, SP6 promoter and T7 promoter from bacteriophage Lamda, lpp promoter, rac5 promoter, amp promoter, and recA promoter, a promoter for kanamycin resistance gene of transposon Tn903 or Tn5, a promoter for metallothionine, a promoter for growth hormone or a hybrid promoter between an eukaryotic and a prokaryotic promoter. In one preferred embodiment, the present vector includes a promoter according to the present disclosure having a nucleic acid sequence as disclosed in SEQ ID NO: 3.

The present vector system can be constructed using various methods known in the art. For example Sambrook et al., Molecular Cloning, A Laboratory Manual, Cold Spring Harbor Laboratory Press (2001) may be mentioned. In one illustrative embodiment, the gene comprised in the present vector is cloned by polymerase chain reaction. In one embodiment, the primer used for cloning the present DNA into a vector is represented by SEQ ID NOs: 5 and 6.

PCR is a widely used method for amplifying nucleic acids molecules and many modifications/variations thereof are known in the art. As examples, a touch down PCR, a hot start PCR, a nested PCR and booster PCR are developed to improve specificity or sensitivity of PCR may be mentioned. Also developed are real time PCR, differential display PCR, rapid amplification of cDNA ends, multiplex PCR, inverse polymerase chain reaction, vectorette PCR and thermal asymmetric interlaced PCR. A detailed explanation on the PCR may be found in M. J., and Moller, S. C. PCR. BIOS Scientific Publishers, Springer-Verlag New York Berlin Heidelberg, N.Y. (2000).

The term “amplification reaction” as used herein refers to a reaction amplifying nucleic acid molecules. Various methods for amplification are known in the art, which for example include PCR (U.S. Pat. Nos. 4,683,195, 4,683,202, and 4,800,159), Reverse Transcription PCR (RT-PCR) (Sambrook et al., ibid), method as disclosed in WO 89/06700 by Miller, H. I. and EP 329,822 by Davey, C.et al., Ligase chain reaction (LCR)(Wiedmann M et al., 1994. PCR Methods Appl), Oap-LCR(WO 90/01069), repair chain reaction (EP 439,182), transcription-mediated amplification (TMA) (WO 88/10315), self-sustained sequence replication (WO90/06995), selective amplification of target polynucleotide sequences) (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909 and 5,861,245) and nucleic acid sequence based amplification (NASBA) (U.S. Pat. Nos. 5,130,238, 5,409,818, 5,554,517 and 6,063,603), but are not limited thereto. Other methods which may be used also described in U.S. Pat. Nos. 5,242,794, 5,494,810, 4,988,617 and application Ser. No. 09/854,317.

Also, the vectors of the present disclosure may be constructed using plasmids including such as for example pMV306, pSC101, ColE1, pBR322, pUC8/9, pHC79, pUC19, pET and the like, and a phage such as for example, λgt4λB, λ-Charon, λΔz1 and M13 and the like, or virus such as for example SV40 and the like, which are known in the art.

In addition, the present vector may further comprise one or more selective markers. In one illustrative embodiment, the present vector may comprise genes encoding a protein conferring resistance to antibiotics, which include, but are not limited to, genes conferring resistance to kanamycin, hygromycin, ampicillin, streptomycin, penicillin, chloramphenicol, gentamicin, carbenicillin, geneticin, neomycin or tetracycline.

In other aspect the present disclosure relates to a transformant containing the present vector. The vectors which may be used for transforming the cells are as described above.

The host cells which may be used for the transformation by the present vector includes any cells known in the art, for example, E. coli DH5α, E. coli JM109, E. coli BL21(DE3), E. coli RR1, E. coli LE392, E. coli B, E. coli X 1776, E. coli W3110, mycobacterial cells.

In one exemplary embodiment, the promoter which may be used for the present vector includes, but is not limited to, the promoter as disclosed in SEQ ID NO: 3, a heat shock protein promoter, a CMV promoter, a promoter for 65 kDa common antigen of mycobacteria, a ribosome RNA promoter from Mycobacteria, a promoter for MPB70, MPB59 or MPB64 antigen from Mycobacterium bovis, tac promoter, trp promoter, lac promoter, IacUV5 promoter, P_{L}^{λ} promoter, P_{R}^{λ}, SP6 promoter and T7 promoter from bacteriophage Lamda, lpp promoter, rac5 promoter, amp promoter, and recA promoter, a promoter for kanamycin resistance gene of transposon Tn903 or Tn5, a promoter for metallothionine, a promoter for growth hormone or a hybrid promoter between an eukaryotic and a prokaryotic promoter.

Methods to deliver the present vector to host cells are known in the art. For example, when the host cells are eukaryotes, CaCl₂ precipitation method (Cohen, S. N. et al., Proc. Natl. Acad. Sci. USA, 9:2110-2114(1973)), Hananhan's method (Hanahan, D., J. Mol. Biol., 166:557-580(1983)) and/or electroporation method (Dower, W. J. et al., Nucleic. Acids Res., 16:6127-6145(1988)) may be used.

The term “mycobacterium” as used herein refers to a population of bacteria classified as gram positive bacteria having a thick and waxy cell wall of hydrophobic enriched with mycolic acid. The mycobacterium is generally classified as non-pathogenic bacteria with a rapid growth property and pathogenic mycobacteria with a slow growth property when cultured. The non-pathogenic mycobacteria include, but are not limited to, a MOTT related to M. intracellulare INT-5, M. yongonense, MOTT-12, MOTT-27, MOTT-64y, M. smegmatis, M. fortuitum, M. parafortuitum, M. vaccae, M. flavescens, M. phlei, M. cuneatum, M. gastri, M. ID-Y, M. neoaurum, M. peregrinum, or M. diernhoferi, M. wolinsky or M. sp. strain JC1 and the like. The non-pathogenic mycobacteria include, but are not limited to, M.tuberculosis, M. bovis, M. leprae, M. marinum, M. avium, M. ulcerans, M. abscessus, M. chelona), M. asiaticum, and M. porcinum and the like. Some mycobacteria are known to use carbon dioxide as their only carbon and energy source (Park S W, Hwang E H, Park H, Kim J A, Heo J, Lee K H, Song T, Kim E, Ro Y T, Kim S W, Kim Y M., J. Bacteriol. 185(1):142-7(2003)). Due to the safety reason and the convenience of the culture, the research on the pathogenic mycobacteria is usually done using the non-pathogenic mycobacteria.

In one illustrative embodiment, the present vector is transformed to mycobacteria. In one embodiment, the mycobacteria includes, but are not limited to, M. smegmatis, M. bovis-BCG, M. avium, M. phlei, M. fortuitum, M. parafortuitum, M. lufu, M. partuberculosis, M. gastri, M. habana, M. scrofulaceum, or M. intracellulare, M. vaccae, M. flavescens, M. cuneatum, M. ID-Y, M. neoaurum, M. peregrinum, or M. diemhoferi.

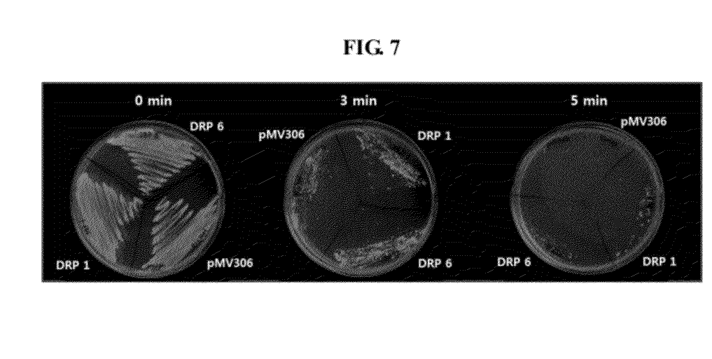

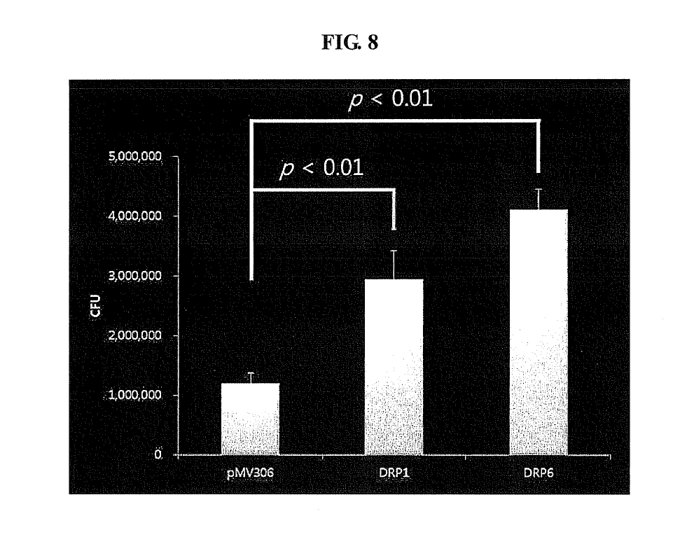

In one illustrative embodiment, the mycobacterial cells transformed with present vector shows resistance to UV and/or macrophages (see FIGS. 7 and 8).

The term “mutation” as used herein refers to changes in a nucleic acid sequences caused artificially or environmentally. Generally the DNA mutation may be caused by various factors such as ultra violet rays, viruses, transposon, mutagens, or by the cell developmental process through for example hypermutation. The mutation may cause various changes in DNA sequences, which for example, include nonsense mutation, frame shift mutation, missense mutation and the like. The non-sense mutation is a point mutation that results in a termination codon. The frame shift mutation is a mutation caused by a deletion or addition of one or more bases and usually results in the changes in the frame of amino acid coding sequences. The missense mutation is a point mutation that results in a change encoding another amino acid, which is often related to the loss of activity of the corresponding protein.

Macrophages are immune cells that remove pathogens infecting a host, and produce various materials to digest and destroy the pathogens engulfed, among which is nitric oxide, NO. However, pathogens that can survive and amplify in macrophages use NO inhibitors to avoid or inhibit the activity of NO or synthesize an enzyme that detoxifies NO produced by the macrophages. The mycobacteria produce enzymes such as NO deoxigenase or peroxynitritase to oxidize NO in macrophages (Couture, M., S. R. Yeh, B. A. Wittenberg, J. B. Wittenberg, Y. Ouellet, D. L. Rousseau, and M. Guertin., Proc. Natl. Acad. Sci. 96:11223-11228(1999); and Wengenack, N. L., Jensen, M. P., Rusnak, F., and Stern, M. K. Biochem.Biophys. Res. Commun. 256:485487(1999)).

In one illustrative embodiment, the mycobacteria transformed with the present vector shows a higher resistance, for example, 3 to 4 times higher resistance to macrophages compared to the controls (FIG. 8).

In other aspect the present disclosure relates to a method of detecting a MOTT (mycobacteria other than tuberculosis) comprising: obtaining a sample containing a nucleic acid molecule; and analyzing the sample for a presence of the isolated DNA molecule of the present disclosure, i.e., MutS4A and MutS4B, wherein the presence of MutS4A and MutS4B indicates the presence of MOTT in the sample.

In still other aspect the present disclosure relates to a kit for diagnosing a disease related to a MOTT comprising a probe and/or a primer set to detect the isolated DNA molecule of the present disclosure.

In the present method, the presence of the isolated DNA molecule of the present disclosure may be detected at the DNA level, mRNA and/or protein expression level. In one embodiment, the presence is performed at the DNA or RNA level using PCR as described herein.

In the present method and kit, the detection is performed by PCR using the primer set which includes a pair of a forward and a reverse primer, each represented by a sequence as disclosed in SEQ ID NO: 5 and 6, respectively.

The term “mycobacteria other than tuberculosis” or “MOTT”, which is also referred as NTM (Nontuberculous mycobacteria), refers to mycobacteria that can cause lung disease similar to tuberculosis, lymphadenitis, skin disease or disseminated disease.

The present method or kit is particularly useful for detecting the presence of M. yongonense, Mycobacteria related to M.intracellulare INT5. In one embodiment, the Mycobacteria related to M.intracellulare INT5 includes MOTT-12, MOTT-27 and MOTT-64y.

The term “sample” as used herein refers to a biological sample derived from mammals, particularly human, and includes, but is not limited to, such as archival tissues, bronchial washes, saliva and blood.

The term “diagnosis or diagnosing” as used herein refers to determining the susceptibility to a particular disease or disorder in a subject, determining the presence of a particular disease or disorder in a subject, or determining a prognosis of a subject having a particular disease or disorder, which for example includes identifying a disease caused by MOTT, or therametrices for such as providing an information for therapeutic efficacy.

The primer and/or probe as may be used for the present method or kit is used for a amplification reaction to amplify and detect a target gene, particularly MutS4A and MutS4B of the present disclosure. The amplification process may be performed using cDNA synthesized from mRNA derived from a biological sample as described above.

The mRNA used for the present method or kit is a total mRNA. The methods to extract the total mRNA from cells or tissues are known in the art (See Sambrook, J. et al., Molecular Cloning. A Laboratory Manual, 3rd ed. Cold Spring Harbor Press(2001); Tesniere, C. et al., Plant Mol. Biol. Rep., 9:242(1991); Ausubel, F. M. at al., Current Protocols in Molecular Biology, John Willey & Sons(1987); and Chomczynski, P. et al., Anal. Biochem. 162:156(1987)). For example, Trizol® reagent may be used for the isolation of total RNA from cells or tissues. The mRNA molecules isolated from biological samples of mammalian origin, have poly-A tails at their 3′ end and thus oligo-dT primers may be used for specific selection of mRNA, which are then transcribed to cDNA molecules using reverse transcriptase (See, PNAS USA, 85:8998(1988); Libert F, et al., Science, 244:569(1989); or Sambrook, J. et al., Molecular Cloning. A Laboratory Manual, 3rd ed. Cold Spring Harbor Press(2001)). The transcribed cDNAs are then amplified by amplification process such as PCR.

The primer or probe used for the present method or kit anneals to the corresponding sequences and forms a double strand hybrid. The condition for the annealing or hybridization may be found in Joseph Sambrook et al, Molecular Cloning, A Laboratory Manual, Cold Spring Harbor Laboratory Press, Cold Spring Harbor, N.Y.(2001); and Haymes, B. D., et al, Nucleic Acid Hybridization, A Practical Approach, IRL Press, Washington, D.C. (1985).

The polymerase known in the art which is used for the PCR may be used for the present disclosure, and includes for example, Klenow fragment of E.coli DNA polymerase I and thermo stable DNA polymerase and T7 DNA polymerase from bacteriophage. In one illustrative example, the polymerase which may be used for the present disclosure includes, but is not limited to, Thermus aquaticus(Taq), Thermus thermophilus(Tth), Thermus filiformis, Thermis flavus, Thermococcus literalis, and Pyrococcus furiosus(Pfu).

For the amplification reaction, it is preferable to use an excess amount of components used in the reaction, which means that the amplification per se is not limited by the components required for the reaction. For example, it is desired to include excess amount of Mg²⁺, and dATP, dCTP, dGTP and dTTP in a reaction to achieve the desired amplification of the sequence of interest. The polymerases used for the reaction is active in identical conditions. The buffer system enables the polymerases to reach the optimal condition for activity. Thus, the amplification process of the present disclosure may be performed in a single reaction without changes such as adding some additives.

The annealing which may be used for the present method or kit is performed on a stringent condition that enables a specific association between a target nucleotide sequence and a primer or a probe. The stringent condition is sequence dependent and varies with other conditions. The amplified products then are analyzed/detected using a proper means and determined for the presence of MOTT. For example, the amplified products may be analyzed by electrophoresis followed by staining and detecting the presence of a band of a particular size.

The detection or diagnosis may also be done by immunoassay based on specific antigen-antibody reaction. In this case, aptamers or antibodies, which specifically bind to the present DNA molecule, MutS4A or MutS4B, are used.

The antibodies which may be used for the present disclosure includes monoclonal or polyclonal antibodies. In one embodiment, monoclonal antibodies are used. Antibodies may be prepared by methods known in the art, for example a fusion method (Kohler and Milstein, European Journal of Immunology, 6:511-519(1976)); recombinant DNA method as disclosed in U.S. Pat. No. 4,816,56; and a method using a phage library of antibodies (Clackson et al, Nature, 352:624-628(1991); Marks et al, J. Mol. Biol., 222:58, 1-597(1991)). The general method to prepare antibodies may be found in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, NY, 1991, the entire content of which are incorporated herein by reference. For example, the preparation of a hybridoma producing an antibody of interest is done by fusing immortal cells with antibody producing lymphocytes, the process of which are known in the art and may be practiced without difficulty by the skilled person in the art. Polyclonal antibodies may be produced by injection a proper antigen into an animal followed by collecting from the animal the serum and isolating the antibody contained therein by using an affinity chromatography.

When the detection/analysis is performed by using antibodies or aptamers, the present method may be used for diagnosing MOTT related disease by detecting the presence of MOTT performed in accordance with the conventional immune assays.

The conventional immune assays may be performed by following a quantitative or qualitative assay protocol known in the art, which includes, but is not limited to a radio-immuno assay, a radio-immuno precipitation assay, a capture-ELISA, a suppression or competitive assay, a sandwich assay, a flow cytometry assay, an immunofluorescence and an immune affinity assay. The details about the assays as described above may be found in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies: A Laboratory Manual, Cold Spring Harbor Laboratory Press, 1999, which are incorporated herein by reference.

In still other aspect, the present disclosure provides a method of using the isolated DNA molecule of the present disclosure to increase a frequency of homologous recombination. The mycobacterial cells transformed with the isolated DNA of the present disclosure showed the increased frequency of recombination. Thus the present mismatch DNA may be useful for increasing the integration frequency of a heterologous gene introduced into mycobacterial cells.

In still other aspect, the present disclosure provides a method of using the isolated DNA molecule of the present disclosure to increase a genetic stability of a heterologous plasmid in cells. The mycobacterial cells transformed with the isolated DNA of the present disclosure conferred a genetic stability to the heterologous gene introduced. Thus the present mismatch DNA may be useful for stable expression of a heterologous plasmid gene in mycobacterial cells.

The present disclosure relates to novel DNA molecules and their uses. The DNA molecules of the present disclosure derived from mycobacteria as classified as MutS4A and MutS4B have a DNA mismatch repair function. Also, the cells transformed with the present DNA shows resistance to UV and/or macrophages and increased homologous recombination frequency. Further the DNA of the present disclosure confers the genetic stability of a heterologous nucleic acid molecule such as plasmid. Therefore the present DNA or the vector comprising the DNA may be useful for functional studies of the genes involved in DNA mismatch repair system and be used for increasing resistance to UV and macrophages and for increasing homologous recombination and genetic stability of a heterologous nucleic acid molecule.

The present disclosure is further explained in more detail with reference to the following examples. These examples, however, should not be interpreted as limiting the scope of the present invention in any manner.

### EXAMPLES

Identification of DNA Mismatch Repair Genes Isolated from a New Mycobacteria

The nucleotide sequences of DNA mismatch repair genes isolated from a new MOTT, M. yongonense, are disclosed as SEQ ID NO: 1 and 2. The nucleotide sequences were analyzed using the BLAST program, which revealed that MutS4A had the highest homology to MutS from Gluconacetobacter diazotrophicus PAI 5 (79% of homology, Table 1), and MutS4B had the highest homology to MutS from Acidothermus cellulolyticus 11 B (67% of homology, Table 2). Also the amino acid sequences encoded by the nucleotide sequences as disclosed in SEQ ID NO: 1 and 2 are represented by SEQ ID NO: 7 and 8, respectively, which are used for the amino acid sequence homology tree as shown in FIG. 1.

TABLE 1
Results of homology search of the present MutS4A amino acid sequence

| Accession | | Max | Total | Query | E- | Max |
|---|---|---|---|---|---|---|
| No. | Genes | Score | Score | coverage | value | homology |
| CP001189.1 | Gluconacetobacter | 105 | 148 | 21% | 6.00E−19 | 79% |
| | diazotrophicus PAI 5, | | | | | |
| | complete genome | | | | | |
| AM889285.1 | Gluconacetobacter | 105 | 105 | 21% | 6.00E−19 | 69% |
| | diazotrophicus PAI 5 | | | | | |
| | complete genome | | | | | |
| CP000481.1 | Acidothermus cellulolyticus | 102 | 102 | 30% | 8.00E−18 | 65% |
| | 11B strain 11B, | | | | | |
| | complete genome | | | | | |
| CP001364.1 | Chloroflexus sp. Y-400-fl, | 93.3 | 93.3 | 20% | 4.00E−15 | 67% |
| | complete genome | | | | | |
| CP000909.1 | Chloroflexus aurantiacus | 93.3 | 93.3 | 20% | 4.00E−15 | 67% |
| | J-10-fl, complete genome | | | | | |

TABLE 2
Results of homology search of the present MutS4B amino acid sequence

| Accession | | Max. | Total | Query | E- | Max |
|---|---|---|---|---|---|---|
| No. | Genes | Score | Score | coverage | Value | Homology |
| CP000481.1 | Acidothermus cellulolyticus | 116 | 116 | 26% | 4.00E−22 | 67% |
| | 11B strain 11B, | | | | | |
| | complete genome | | | | | |
| AP011532.1 | Methanocella paludicola | 104 | 104 | 19% | 2.00E−18 | 68% |
| | SANAE DNA, | | | | | |
| | complete genome | | | | | |
| CP002042.1 | Meiothermus silvanus DSM | 98.7 | 98.7 | 16% | 1.00E−16 | 68% |
| | 9946, complete genome | | | | | |
| CP002021.1 | Thiomonas intermedia K12, | 78.8 | 145 | 23% | 9.00E−11 | 81% |
| | complete genome | | | | | |
| FP475956.1 | Thiomonas sp. str. 3As | 71.6 | 137 | 24% | 1.00E−08 | 82% |
| | chromosome, complete genome | | | | | |
| CP001839.1 | Thermotoga naphthophila | 55.4 | 55.4 | 6% | 0.001 | 72% |
| | RKU-10, complete genome | | | | | |

### Example 2

### Primer Pair for PCR Targeting the Mismatch Repair Gene from Mycobacteria

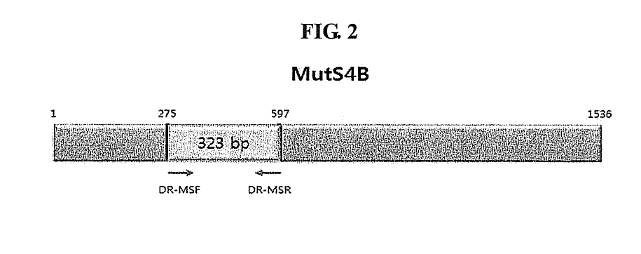

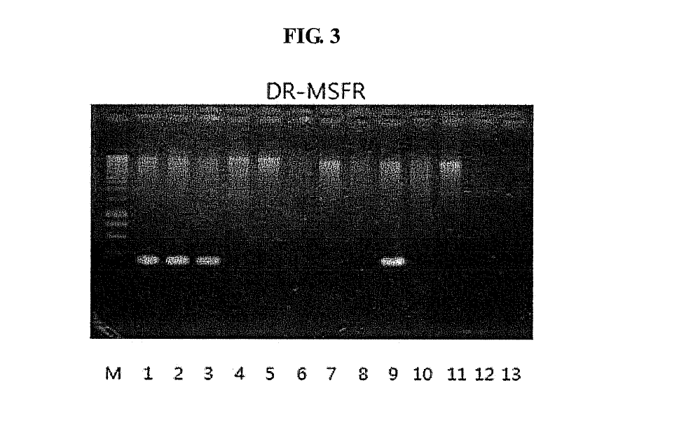

To identify other MOTT having the isolated DNA of Example 1, the primer pairs were designed as follows. The primer pair was designed to amplify a region of MutS4B having a total of 1536 bases in length corresponding to 275th nucleotide to 597th nucleotide generating a product of 323 bp in length (FIG. 2). The primer sequences are as follows: the sense primer (DR-MSF); 5′-TCCAGGTCCGGCGCAAGGTGTT-3′ (SEQ ID NO: 5) and the reverse primer (DR-MSR); 5′-CGCGGGCGGCTGATGAAGAAGATA-3′ (SEQ ID NO: 6). The results from PCR with the primer pair as described above using various MOTTs isolated from patients as a template is indicated in FIG. 3.

The primers detected MOTT, particularly Mycobacterium related to Mycobacterium Intracellulare INT-5 (Park et al., Molecular characterization of Mycobacterium intracellulare-related strains based on the sequence analysis of hsp65, internal transcribed spacer and 16S rRNA genes. J Med Microbiol. 2010 September: 59 (Pt 9):1037-43), M. yongonense (Kim et al., Mycobacterium yongonense sp. nov., a novel slow growing nonchromogenic species closely related to Mycobacterium intracellulare. Int J Syst Evol Microbiol. 2012 Mar. 16. [Epub ahead of print] PMID:22427442) MOTT-12 and MOTT-27 which were identified by the present inventors.

### Example 3

### Cloning of the DNA Mismatch Repair Gene from a Novel Mycobacterial DNA

About 4kb of DNA fragment having the DNA mismatch repair gene and a putative promoter was amplified using a primer pair: sense primer; 5′-TTGCGGCCGCCGACCGAGTTGGCGTGG-3′ and antisense primer; 5′-CTGACTGCCGTCTAAAGGTCTAGAGC-3′. The underlined sequence of the sense and antisense primer indicates Notl and Xbal restriction site, respectively. The amplified product was confirmed by sequencing and the sequence is disclosed as SEQ ID NO: 4.

The PCR amplified product thus obtained using the primer pair as described above were digested with Notl and Xbal and ligated to pMV306 vector that was also digested with the same enzyme. The pMV306 is a vector capable of integrating the gene encoded therein into the genome of mycobacteria using an integrase system of mycobacterial phage L5 [7,8; FIG. 5]

### Example 4

### Transformation of Mycobacterium smegmatis MC2-155 with the DNA Mismatch Repair Gene

The competent cells of Mycobacterium smegmatis MC2-155 was transformed with pMV306 vector comprising the DNA mismatch repair gene constructed as described in Example 3 by electroporation (2.5 kV, 1,0000 and 2,500 pF). After the electroporation, the cells were cultured in Middlebrook 7H9 broth (Difco, USA) containing 10% ADC(Albumin-Dextrose-Catalase) while shaking and plated on Middlebrook 7H9 agar plate containing 10% OADC (Oleic acid-Albumin-Dextrose-Catalase; Difco) and 100 ,μg/ml of kanamycin and allowed to form colonies by incubating the plates at 37° C.

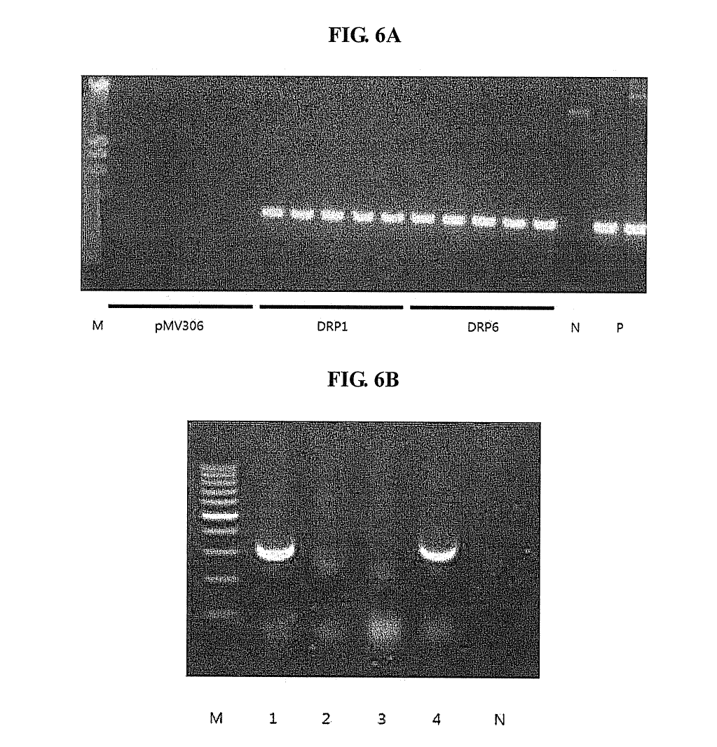

The colonies were picked and PCRs were performed using DR-MSF DR-MSR primer pair as described above. As a result, it was confirm that the DNA mismatch repair gene was integrated into the genome of the transformants (FIG. 6A) . Also the expression from the integrated gene was confirmed by a reverse transcription PCR using the same primer as described above as shown in FIG. 6B.

### Example 5

### Resistance to UV and Macrophages by the Mycobacteria Transformed with the Mismatch Repair DNA

To test the UV resistance conferred by the present DNA mismatch repair gene, mycobacteria were transformed with an empty vector (negative control) or the vector comprising the DNA mismatch repair gene as prepared in Example 3 as described in Example 4. The cells were then streaked on 7H10 agar plates and illuminated with UV for 0, 3, and 5 min followed by incubation at 37 for 2-3 days. As shown in FIG. 7, the mycobacteria transformed with the DNA mismatch repair gene showed a higher UV resistance compared to the negative control.

To test the resistance to macrophage, macrophage cell line, J774A.1(ATCC TIB-67) cells derived from mouse were used for the infection test. J774A.1 cells at the concentration of 1×10⁵ cells/well were added to a well of a 24 well plate and incubated overnight at 37° C. After the incubation, Mycobacterium smegmatis-pMV306 strain and Mycobacterium smegmatis-DNA mismatch repair gene strain were suspended on a DMEM (Thermo, USA) medium without antibiotics at the concentration of 1×10⁶ CFU(colony forming units) and added to the wells plated with J774A.1 at the multiplicity of infection(MOI)=10 and incubated for 1 hour. After that, the cells in each well were washed with PBS and incubated for 2 hours after DMEM medium containing 10 μg/ml of gentamycin (Sigma, USA) was added. After 2 hours of incubation, the medium was replaced with fresh DMEM without antibiotics. Two days after the infection, the cells were harvested in the PBS containing 0.5% Triton® X-100(Merck, USA) and diluted 1/100 or 1/1000 and plated on 7H10 agar plate containing 100 μg/ml of kanamycin. The colonies formed were counted after 3 days. As shown in FIG. 8, the strain containing the DNA mismatch repair gene formed 3-4 times more colonies than the strain containing only the empty vector pMV306.

### Example 6

### Frequency of Homologous Recombination in the Transformed M. smegmatis Strain

RNA polymerase beta subunit coding fragment (684bp) from M. tuberculosis was amplified by PCR using M. tuberculosis genomic DNA (with mutation at rpoB codon 522 a.a (317) and 526 a.a (309)) as template and Forward: BamHI-TB-rpoB-F: 5′-CGG GAT CCC GTC GGT CGC TAT AAG GTC AAC A-3′ and Reverse: HindIII-TB-rpoB-R: 5′-CCC MG CTT CTC GTC GGC GGT CAG GTA-3′ as primers with the following condition: 5 min at 95° C.; 30 cycles of 30 sec at 95° C.; 30 sec at 63° C.; 45 sec at 72° C.; 5 min at 72° C.

The amplified fragment was then cloned into the BamHI and Hindi!l sites of pSE100 (www.addgene.org) to construct pSE100-309 and-317.

The pSE100 is a vector that is normally used as mycobacteria-E.coli shuttle vector. The pSE100-309 and-317 constructed each contains a genetic mutation in rpoB gene at codon 526; CAC→TAC and 522; TCG→TTG, respectively, which confers resistance to rifampin in cells

Each of pSE100-309 and-317 vector was then introduced into each of M. smegmatis strain transformed with the present DNA mismatch repair gene or an empty vector pMV306 as described in Example 4. The cells were then plated on 7H10 +hygromycin (50 μg/ml) agar plate and incubated for 72 hours at 37° C. The colonies formed were picked and suspended in 7H9+hygromycin (50 μg/ml) liquid media and cultured for 72 hours at 37° C. The cells were then adjusted to OD600 of 0.2 and plated on the 7H10 +rifampin (100 μg/ml) agar plate. The number of colonies formed was counted and the rpoB gene was amplified by PCR using primers 7940F (5′-TCAAGG AGA AGC GCT ACG ACC-3′) and MR (5′-TCG ATC GGG CAC ATC CGG-3′) from the colonies and its nucleotide sequence was determined.

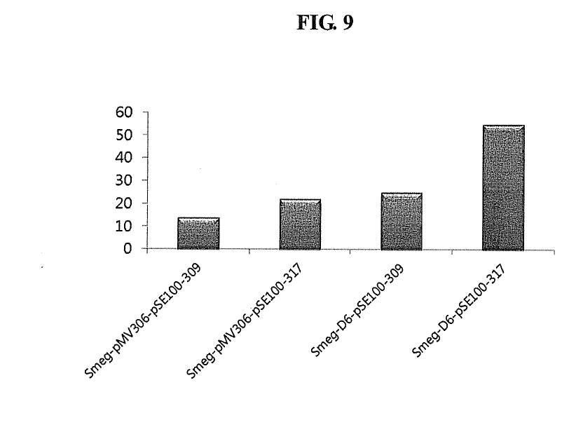

As shown in FIG. 9, more colonies were formed from the M. smegmatis strain transformed with the present DNA mismatch repair gene (Smeg-D6) than those from M. smegmatis strain transformed with the empty vector pMV306.

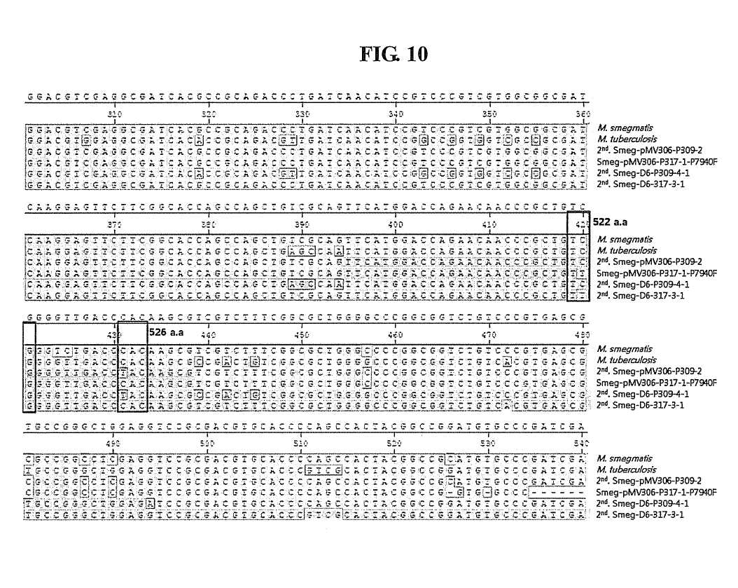

Also as shown in FIG. 10, the multiple sequence alignment of rpoB genes as sequenced above showed that the M. smegmatis strain transformed with the present DNA mismatch repair gene (Smeg-D6) had the frequency of homologous recombination and the length of putative nucleic acid sequences that underwent recombination superior to those of the cells transformed with an empty vector pMV306.

### Example 7

### Genetic Stability of the Exogenous Plasmid in the Transformed Cells

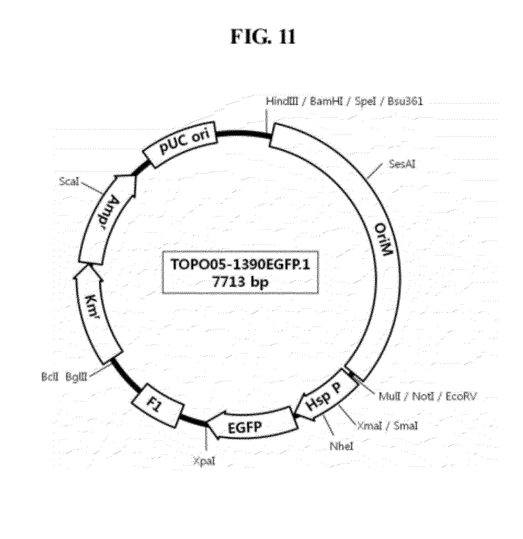

Each of M. smegmatis strain transformed with the present DNA mismatch repair gene or an empty vector pMV306 as described in Example 4 was transformed with a mycobacteria-E.coli shuttle vector comprising EGFP under the control of heat shock protein promoter developed by the present inventors as indicated in FIG. 11. The cells were passaged 5 times, and the subcultured cells of each passage were analyzed by FACS for the expression of EGFP protein.

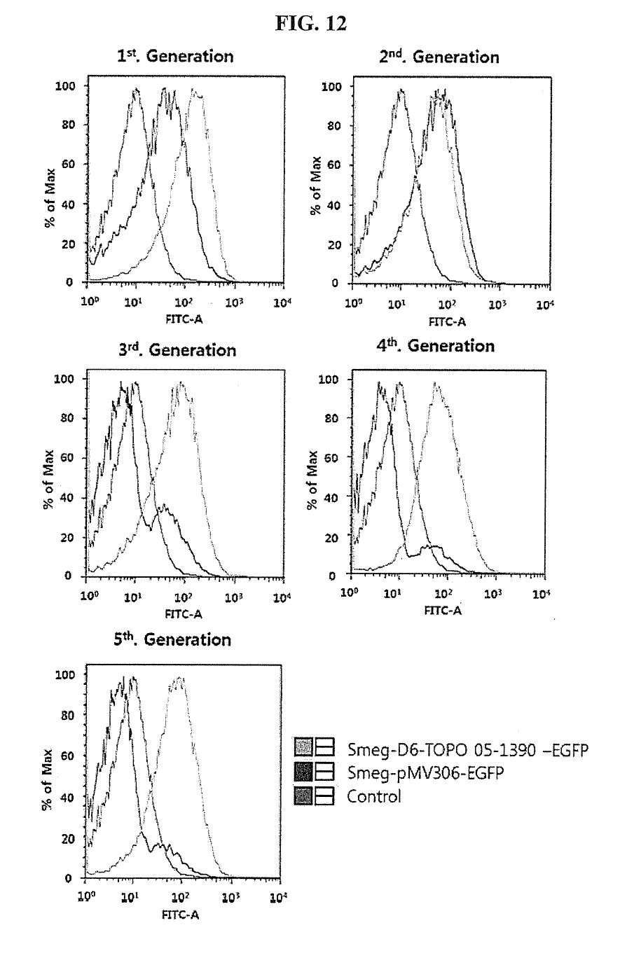

As shown in FIG. 12, with the increase passage number, M. smegmatis strain transformed with the empty vector pMV306 showed dramatic decrease in the expression level of EGFP. This is in contrast to the result obtained with the cells transformed with the present DNA mismatch repair gene, where the expression level of EGFP was stably maintained.

These results indicate that the isolated DNA of the present disclosure increase the genetic stability of the heterologous genes such plasmid.

With respect to the use of substantially any plural and/or singular terms herein, those having skill in the art can translate from the plural to the singular and/or from the singular to the plural as is appropriate to the context and/or application.

The various singular/plural permutations may be expressly set forth herein for sake of clarity. Although a few embodiments of the present disclosure have been shown and described, it would be appreciated by those skilled in the art that changes may be made in this embodiment without departing from the principles and sprit of the invention, the scope of which is defined in the claims and their equivalents.

What is claimed is:

1. An isolated DNA molecule for DNA mismatch repair derived from Mycobacteria having a nucleic acid sequence as disclosed in SEQ ID NO: 1 or 2.

2. A promoter having a nucleic acid sequence as disclosed in SEQ ID NO: 3.

3. A recombinant vector comprising (i) the DNA molecule according to claim 1; and (ii) a promoter operatively linked to the DNA.

4. The recombinant vector according to claim 3, wherein the vector is pMV306 comprising the DNA molecule according to claim 1 and the promoter according to claim 2.

5. The vector according to claim 3, wherein the promoter includes a promoter according to claim 2, a heat shock protein promoter, a CMV promoter, a promoter for 65 kDa common antigen of mycobacteria, a ribosome RNA promoter from Mycobacteria, a promoter for MPB70, MPB59 or MPB64 antigen from Mycobacterium bovis, tac promoter, trp promoter, lac promoter, lacUV5 promoter, P_{L}^{λ} promoter, P_{R}^{λ}, SP6 promoter and T7 promoter from bacteriophage Lamda, lpp promoter, racy promoter, amp promoter, and recA promoter, a promoter for kanamycin resistance gene of transposon Tn903 or Tn5, a promoter for metallothionine, a promoter for growth hormone or a hybrid thereof.

6. The vector according to claim 3, wherein the vector further includes a selection marker gene.

7. A cell transformed with the vector according to claim 3.

8. The cell according to claim 7, wherein the cell is Mycobacteria.

9. The cell according to claim 8, wherein the Mycobacteria includes M. smegmatis, M. bovis-BCG, M. avium, M. phlei, M. fortuitum, M. parafortuitum, M. lufu, M. partuberculosis, M. gastri, M. habana, M. scrofulaceum, or M. intracellulare, M. vaccae, M. flavescens, M. cuneatum, M. ID-Y, M. neoaurum, M. peregrinum, or M diernhoferi.

10. The cell according to claim 7, wherein the cell shows resistance to UV and/or macrophage.

11. A method for detecting a MOTT (mycobacteria other than tuberculosis) comprising the steps of:
obtaining a sample containing a nucleic acid molecule; and
analyzing the sample for the presence of the DNA molecule according to claim 1, MutS4A and MutS4B, wherein the presence of MutS4A and MutS4B indicates the presence of MOTT in the sample.

12. The method according to claim 11, wherein the sample is at least one of archival tissues, bronchial washes, saliva and/or blood.

13. The method according to claim 11, wherein the analysis is performed by using a polymerase chain reaction.

14. The method according to claim 11, wherein the MOTT is a MOTT related to M. intracellulare INT-5, M. yongonense, MOTT-12, MOTT-27, and/or MOTT-64y.

15. A kit for diagnosing a disease related to a MOTT comprising a probe and/or a primer set to detect the DNA molecule according to claim 1.

16. The kit according to claim 15, wherein the primer set includes a first and a second primer, each represented by a sequence as disclosed in SEQ ID NO: 5 and 6, respectively.

17. The kit according to claim 15, wherein the detection is performed by using a polymerase chain reaction.

18. The kit according to claim 15, wherein the MOTT is a MOTT related to M. intracellulare INT-5, M. yongonense, MOTT-12, MOTT-27, and/or MOTT-64y.

19. A method of using the isolated DNA molecule according to claim 1 to increase a frequency of homologous recombination.

20. A method of using the isolated DNA molecule according to claim 1 to increase a genetic stability of a heterologous plasmid in a cell.


SEQUENCE LISTING (8 sequences)

SEQ ID NO: 1 - DNA, 1533 bp, M.yongonense
atggccatcttcaccagcatccttgaccccggagtggatgaccgcggcagtagctcgccaccctcaccagacttcctcgccgaccttcatctcgaccagatcttcgccgcggtgacggctgggtatggcgacagtgagatcgcgagtactttctgtgcacccctccatgacctgcgggctgtgcagtaccgccagcaagtgttccgtgacctcgaggacgagcacacacggtcatcgatacaaaacttcgtcgacggcatccgtgccatgcgcggtcgacttaccgtggcgaggaacgcgtggcaccgcctgcaacgacagggttggctaatcgctgcgatcgagatttattgccgcacaatagaactgctaggcaataatgtcgccgacatgagaatgcgttcgccgggcttgcgcaacttcgctgagcacgttacgggctacatcaatggcgaggctttcaagacgctggccgccgagacccaaacgatgcgagaccatctgcgcagagtgcgatacatggtgcacattgagggccttcaagttcatgtccaaaagttcaacgaacaaagtgattacagtacggccatcgctgaaacgtttgaacgtttcgcgacggaggtcagcaaggactaccgtgtcgcgcttccggaattcaacgatatgaaccatatcgaggagcagattctcgaatgcgtcgctagacttcatccggaagcctttaggcttctcgatagcttctgccacaatcacgaacactttatagaaccgattgttgcgagatttgagcacgagatccacttctatctctcatatctgatgttcatcggccgtttcaggacgctgggactcgcgttttgttatccggacatgaccgacgatcccggcgtgatgaatgttggcgacgctttcgaccttgcgctggccatcagtatggtccaggaacaaaatcgccctgtcaccaacgacttctatctgtcgggatccgaacgcaccttcgtcgttaccggcccaaaccaggggggcaaaacgacgttcgcccggacgatcggccagtgcgcttacctcgcgtcgattggctgtccgatacctgccagcgtcgcccgcctgaccttgcccgaccagatctttacccacttcgagcgacaagaagatctctcgacgctgcatggaaaactcgatgacgagctggtgcgcattcatgacattctctcgcgcgcgaccgataccagcatcctagtcatgaacgaaagcttctcgtcgaccaccgtcaacgatgccttgttaattggtcgagaggtgctcgaacgaatcgtcacactgggttgcatcgcggtctatgtcagtttcctcgacgaactttccacactggatccgatgtgcgtcagcatggttggtgaagtcgctcgagatgacccgacccagcggacctttaagttcactcgccgtcccgctgacggcctggcatatgctgctgcgctcgccaacaaatacggcttgagctacgacgcactggggcggaggatcagccaa

SEQ ID NO: 2 - DNA, 1533 bp, M.yongonense
atgagagtccggctcctagacccggttcacgaccccgatctattaccgcggctgccttggcgcttgcaggacctcgtcgatgaggacctcgaactgcggcgcgtttacaacgccatggccggcggcgacgactttttactcgagacagcgaaaaaaattgtgccgctttcggttaccgatcctgacgtcatagtctaccgccaacaagtcctggcggattgtctcgccaaccgcgcggtagtgcagcatatgtacgacatcgcggtcgacggcgtccaggtccggcgcaaggtgttcctcgggggattaatgtcccgggacccgcaagccatactgcgccgctccgtccgtgtcctcgaactattagtaggcaaccttcgacagctgcgggcagtatgcagcgaacaaggccatggtttcagctcggtcgggtttcgacagttgcttgacatgatcagcactcaggtctgcgacgagtacctcaagcagctcgatgcagaccttcgcgagctgcatctcccgcgaggggtaatgctcagcgcttcactgagtctgggcaacaagggcacagattatcttcttcatcagccgcccgcgcgcaactggtgggacagattgaccggaaaccacagtggcagctgtggattcgtcgttgatgaccgcgacgaggccggcgcgaaggcgctcaccgaattggccggtcgtgccatcaacgacgtcgccaacacggtcacacgatccgcggaccacgtcgagggcttcttcggccgcttacgcaccgaactcggcttctatttagcatgtaccaacctctacgaacaactcacgaaagcagacgcacccacctgctttccgacccccataccagtaggcctgcctcagttctcttgccgtgatctgcgcgatgtgggcttgtgccttaccggcacaagtccagtcgtcggaaacgacatcgatgctgccgggaggacgttgatcgtgataacgggggctaatgaaggcggtaagtcgacctgcttacgcagcatgggtgtcgcgcaggtgatgatgcaagctggcatgtttgttgccgcggcctcgtttcatgccaacgtccgcgacggtgtcttcacacacttcaaacgcgaagaagacgacaccaacgtccgcggcaagctcgaagaagaacttgctcgcatgagtgacatcgccgacgtggttggctcgacatcattgttgttgtgcaatgagtcctttgcctcgaccaatgagagggaggggtctcagatcgcacgcgatgtaatacgagccatggtggaaaacggggtcaaaatcgtgtttgtgactcatttgtacggcctcgcgcgtcacctcagttcgcaaggcgatccgagctatctcttcttgcgggcccaacgtcgcgacgacggcttacgaacgttccgattaaagccgggacacccggagcccaccagctatggtcaggactcatttcggcgggtcttcggcaacgtcccacaacgtgaggccatctccgaaatcgtgggatccgat

SEQ ID NO: 3 - DNA, 770 bp, M.yongonense
  promoter (1)..(770)
agaaagcagaccgcgcgtcgcacctacgcagctagatttctcactggaccgttgaagtcagcgcacggcgacttgcaagcagccgggccgagtcgaccgagttggcgtgggatcgctacaacgttgtcgccgaatgactctggcctgtagcaggcttgtggttgaggtaggtcatcagaacgtcttccgcacgatggtattgcagtgaatccacccacagtcgccgagtcggatgccgccgcaaattgcgactcagcgttccgcccggggttgccacatacccgccgatgcctgcatccgcgagtcgatccagcacagggccgacgtcgctagcgtcaaggtcggccaactctgcccaatacgctgcaaaaatctcattgtcgataccgcccggcggtagccaaaacatgacgttgtaccgcgccggattccccatcccgcggcatggagaactcatcgacatttccttagctttgagatgggcattgcatcctcgctggtagctacccaggagccattggcgacaatccgcgattcggcgagctccaccgccgcatctcccatcatcacccgcagaagatgagtacgccagggctcttgtgcgacacgacgaaccccacaacaatcgccgtcagacagctcgctgcgccagctgcgcaggtgatctcggggtaacatggacaacacggcgatgaagctcgccacaactgccgcgccggctgatggcttctaccatggacgacttacggtagaaggcgggtgagcgcaag

SEQ ID NO: 4 - DNA, 3838 bp, M.yongonense
agaaagcagaccgcgcgtcgcacctacgcagctagatttctcactggaccgttgaagtcagcgcacggcgacttgcaagcagccgggccgagtcgaccgagttggcgtgggatcgctacaacgttgtcgccgaatgactctggcctgtagcaggcttgtggttgaggtaggtcatcagaacgtcttccgcacgatggtattgcagtgaatccacccacagtcgccgagtcggatgccgccgcaaattgcgactcagcgttccgcccggggttgccacatacccgccgatgcctgcatccgcgagtcgatccagcacagggccgacgtcgctagcgtcaaggtcggccaactctgcccaatacgctgcaaaaatctcattgtcgataccgcccggcggtagccaaaacatgacgttgtaccgcgccggattccccatcccgcggcatggagaactcatcgacatttccttagctttgagatgggcattgcatcctcgctggtagctacccaggagccattggcgacaatccgcgattcggcgagctccaccgccgcatctcccatcatcacccgcagaagatgagtacgccagggctcttgtgcgacacgacgaaccccacaacaatcgccgtcagacagctcgctgcgccagctgcgcaggtgatctcggggtaacatggacaacacggcgatgaagctcgccacaactgccgcgccggctgatggcttctaccatggacgacttacggtagaaggcgggtgagcgcaagatggccatcttcaccagcatccttgaccccggagtggatgaccgcggcagtagctcgccaccctcaccagacttcctcgccgaccttcatctcgaccagatcttcgccgcggtgacggctgggtatggcgacagtgagatcgcgagtactttctgtgcacccctccatgacctgcgggctgtgcagtaccgccagcaagtgttccgtgacctcgaggacgagcacacacggtcatcgatacaaaacttcgtcgacggcatccgtgccatgcgcggtcgacttaccgtggcgaggaacgcgtggcaccgcctgcaacgacagggttggctaatcgctgcgatcgagatttattgccgcacaatagaactgctaggcaataatgtcgccgacatgagaatgcgttcgccgggcttgcgcaacttcgctgagcacgttacgggctacatcaatggcgaggctttcaagacgctggccgccgagacccaaacgatgcgagaccatctgcgcagagtgcgatacatggtgcacattgagggccttcaagttcatgtccaaaagttcaacgaacaaagtgattacagtacggccatcgctgaaacgtttgaacgtttcgcgacggaggtcagcaaggactaccgtgtcgcgcttccggaattcaacgatatgaaccatatcgaggagcagattctcgaatgcgtcgctagacttcatccggaagcctttaggcttctcgatagcttctgccacaatcacgaacactttatagaaccgattgttgcgagatttgagcacgagatccacttctatctctcatatctgatgttcatcggccgtttcaggacgctgggactcgcgttttgttatccggacatgaccgacgatcccggcgtgatgaatgttggcgacgctttcgaccttgcgctggccatcagtatggtccaggaacaaaatcgccctgtcaccaacgacttctatctgtcgggatccgaacgcaccttcgtcgttaccggcccaaaccaggggggcaaaacgacgttcgcccggacgatcggccagtgcgcttacctcgcgtcgattggctgtccgatacctgccagcgtcgcccgcctgaccttgcccgaccagatctttacccacttcgagcgacaagaagatctctcgacgctgcatggaaaactcgatgacgagctggtgcgcattcatgacattctctcgcgcgcgaccgataccagcatcctagtcatgaacgaaagcttctcgtcgaccaccgtcaacgatgccttgttaattggtcgagaggtgctcgaacgaatcgtcacactgggttgcatcgcggtctatgtcagtttcctcgacgaactttccacactggatccgatgtgcgtcagcatggttggtgaagtcgctcgagatgacccgacccagcggacctttaagttcactcgccgtcccgctgacggcctggcatatgctgctgcgctcgccaacaaatacggcttgagctacgacgcactggggcggaggatcagccaatgagagtccggctcctagacccggttcacgaccccgatctattaccgcggctgccttggcgcttgcaggacctcgtcgatgaggacctcgaactgcggcgcgtttacaacgccatggccggcggcgacgactttttactcgagacagcgaaaaaaattgtgccgctttcggttaccgatcctgacgtcatagtctaccgccaacaagtcctggcggattgtctcgccaaccgcgcggtagtgcagcatatgtacgacatcgcggtcgacggcgtccaggtccggcgcaaggtgttcctcgggggattaatgtcccgggacccgcaagccatactgcgccgctccgtccgtgtcctcgaactattagtaggcaaccttcgacagctgcgggcagtatgcagcgaacaaggccatggtttcagctcggtcgggtttcgacagttgcttgacatgatcagcactcaggtctgcgacgagtacctcaagcagctcgatgcagaccttcgcgagctgcatctcccgcgaggggtaatgctcagcgcttcactgagtctgggcaacaagggcacagattatcttcttcatcagccgcccgcgcgcaactggtgggacagattgaccggaaaccacagtggcagctgtggattcgtcgttgatgaccgcgacgaggccggcgcgaaggcgctcaccgaattggccggtcgtgccatcaacgacgtcgccaacacggtcacacgatccgcggaccacgtcgagggcttcttcggccgcttacgcaccgaactcggcttctatttagcatgtaccaacctctacgaacaactcacgaaagcagacgcacccacctgctttccgacccccataccagtaggcctgcctcagttctcttgccgtgatctgcgcgatgtgggcttgtgccttaccggcacaagtccagtcgtcggaaacgacatcgatgctgccgggaggacgttgatcgtgataacgggggctaatgaaggcggtaagtcgacctgcttacgcagcatgggtgtcgcgcaggtgatgatgcaagctggcatgtttgttgccgcggcctcgtttcatgccaacgtccgcgacggtgtcttcacacacttcaaacgcgaagaagacgacaccaacgtccgcggcaagctcgaagaagaacttgctcgcatgagtgacatcgccgacgtggttggctcgacatcattgttgttgtgcaatgagtcctttgcctcgaccaatgagagggaggggtctcagatcgcacgcgatgtaatacgagccatggtggaaaacggggtcaaaatcgtgtttgtgactcatttgtacggcctcgcgcgtcacctcagttcgcaaggcgatccgagctatctcttcttgcgggcccaacgtcgcgacgacggcttacgaacgttccgattaaagccgggacacccggagcccaccagctatggtcaggactcatttcggcgggtcttcggcaacgtcccacaacgtgaggccatctccgaaatcgtgggatccgattga

SEQ ID NO: 5 - DNA, 22 bp, Artificial Sequence
  Synthetic forward primer
tccaggtccggcgcaaggtgtt

SEQ ID NO: 6 - DNA, 24 bp, Artificial Sequence
  DR-MSR reverse primer
cgcgggcggctgatgaagaagata

SEQ ID NO: 7 - protein, 511 aa, M.yongonense
MAIFTSILDPGVDDRGSSSPPSPDFLADLHLDQIFAAVTAGYGDSEIASTFCAPLHDLRAVQYRQQVFRDLEDEHTRSSIQNFVDGIRAMRGRLTVARNAWHRLQRQGWLIAAIEIYCRTIELLGNNVADMRMRSPGLRNFAEHVTGYINGEAFKTLAAETQTMRDHLRRVRYMVHIEGLQVHVQKFNEQSDYSTAIAETFERFATEVSKDYRVALPEFNDMNHIEEQILECVARLHPEAFRLLDSFCHNHEHFIEPIVARFEHEIHFYLSYLMFIGRFRTLGLAFCYPDMTDDPGVMNVGDAFDLALAISMVQEQNRPVTNDFYLSGSERTFVVTGPNQGGKTTFARTIGQCAYLASIGCPIPASVARLTLPDQIFTHFERQEDLSTLHGKLDDELVRIHDILSRATDTSILVMNESFSSTTVNDALLIGREVLERIVTLGCIAVYVSFLDELSTLDPMCVSMVGEVARDDPTQRTFKFTRRPADGLAYAAALANKYGLSYDALGRRISQ

SEQ ID NO: 8 - protein, 511 aa, M.yongonense
MRVRLLDPVHDPDLLPRLPWRLQDLVDEDLELRRVYNAMAGGDDFLLETAKKIVPLSVTDPDVIVYRQQVLADCLANRAVVQHMYDIAVDGVQVRRKVFLGGLMSRDPQAILRRSVRVLELLVGNLRQLRAVCSEQGHGFSSVGFRQLLDMISTQVCDEYLKQLDADLRELHLPRGVMLSASLSLGNKGTDYLLHQPPARNWWDRLTGNHSGSCGFVVDDRDEAGAKALTELAGRAINDVANTVTRSADHVEGFFGRLRTELGFYLACTNLYEQLTKADAPTCFPTPIPVGLPQFSCRDLRDVGLCLTGTSPVVGNDIDAAGRTLIVITGANEGGKSTCLRSMGVAQVMMQAGMFVAAASFHANVRDGVFTHFKREEDDTNVRGKLEEELARMSDIADVVGSTSLLLCNESFASTNEREGSQIARDVIRAMVENGVKIVFVTHLYGLARHLSSQGDPSYLFLRAQRRDDGLRTFRLKPGHPEPTSYGQDSFRRVFGNVPQREAISEIVGSD